(19) United States
(12) Patent Application Publication
(10) Pub. No.: US 2024/0065961 A1
(43) Pub. Date: Feb. 29, 2024

(54) COMPOSITION FOR CARING FOR KERTATIN MATERIALS

(71) Applicant: L'OREAL, Paris (FR)

(72) Inventors: Chi MA, Shanghai (CN); Xiuxia WANG, Shanghai (CN)

(73) Assignee: L'OREAL, Paris (FR)

(21) Appl. No.: 18/259,896

(22) PCT Filed: Dec. 31, 2020
(86) PCT No.: PCT/CN2020/142071

§ 371 Date: Jun. 29, 2023

(51) Int. Cl. A61K 8/60; A61K 8/34; A61K 8/67; A61K 8/9789; A61Q 19/02; A61Q 19/08

(52) U.S. Cl. CPC A61K 8/602; A61K 8/345; A61K 8/347; A61K 8/676; A61K 8/9789; A61Q 19/02; A61Q 19/08; A61K 2800/31; A61K 2800/522

(57) ABSTRACT

Provided herein is a composition comprising A) a component comprising polydatin; and B) at least two polyols, wherein at least one of the polyols is a C2-C6 diol, and wherein the composition is anhydrous. Also provided herein is a process for the care or make-up of keratin materials, comprising the application to the keratin materials of said composition.

### TECHNICAL FIELD

The present invention relates to a cosmetic composition for caring for keratin materials, and in particular the skin. Further, the present invention relates to use of the same, and in particular as a skincare product.

### BACKGROUND

It has always been an ultimate target of the cosmetic filed to deliver products with skin benefits such as hydration, moisturizing, anti-aging, whitening, cleansing, and so on.

With aging, the outer skin layer (epidermis) thins, even though the number of cell layers remains unchanged. The number of pigment-containing cells (melanocytes), however, decreases. Therefore, the skin appears pale and translucent. Large pigmented spots (age spots, liver spots, or lentigos) may appear in sun-exposed areas. Changes in the connective tissue reduce the skin's strength and elasticity. This is known as elastosis. It is more noticeable in sun-exposed areas (solar elastosis). Elastosis produces the leathery, weather-beaten appearance common to farmers, sailors, and others who spend a large amount of time outdoors. Dehydration increases the risk of skin injury. Poor nutrition can also negatively impact the skin, causing dryness, rash, and puffiness.

On the other hand, the formation of free radicals is a widely accepted pivotal mechanism leading to skin aging. Free radicals are highly reactive molecules with unpaired electrons that can directly damage various cellular membranes, lipids, proteins, RNA and DNA. The damaging effects of these reactive oxygen species are induced internally during normal metabolism and externally through various oxidative stresses. UV exposure and environmental pollution can accelerate skin aging by producing free radicals in skin. Antioxidants protect cells from the damage of oxidative stress by scavenging free radicals and inhibiting following oxidation reactions. The topical application of antioxidants is broadly used in skin care products to prevent skin aging.

### SUMMARY OF THE INVENTION

The instant disclosure relates to compositions and methods of using the compositions to benefit skin. The compositions improve the appearance of skin and provide multiple anti-aging and other benefits to the skin. The compositions are unique in their ability to photostabilize polydatin.

The inventors have unexpectedly discovered that an anhydrous composition comprising, consisting essentially of, or consisting of components A) polydatin; and B) at least two polyols, wherein at least one of the polyols is a C2-C6 diol.

According to an embodiment of the invention, component A), i.e., polydatin, is used in an amount of 0.1-18% by weight relative to the total weight of the composition.

According to an embodiment of the invention, the component A) and the component B) are used in a ratio by weight of 0.0001:1 to 0.3:1, preferably 0.0005:1 to 0.25:1, or preferably 0.001:1 to 0.22:1.

The composition according to the invention has in particular a fluid texture, suitable for use in ampoule. The composition according to the invention is especially stable over time, especially against UV radiation.

The subject of the invention is also a process for the care or makeup of keratin materials, in particular skin, e.g., face, comprising the topical application to the keratin materials of the composition according to the present invention.

The invention also relates to the use of the composition according to the present invention for the care or makeup of keratin materials, particularly for the care of the skin. The composition according to the present invention shows particularly an improved anti-singlet oxygen effect. Accordingly, the composition according to the present invention, particularly in the case of being used in an ampoule product, is superior on radiant bright skin and/or smooth nourished skin.

### EMBODIMENTS OF INVENTION

Throughout the description, including the claims, the term “comprising a” should be understood as being synonymous with “comprising at least one”, unless otherwise mentioned. Moreover, the expression “at least one” used in the present description is equivalent to the expression “one or more”.

Throughout the description, including the claims, an embodiment defined with “comprising” or the like should be understood to encompass a preferable embodiment defined with “consisting substantially of” and a preferable embodiment defined with “consisting of”.

Preferably, the “keratin material” according to the present invention is the skin. By “skin”, we intend all the body skin. Still preferably, the keratin material is the face, or the neck, especially the face.

By “topical application”, it meant that the composition is applied or spread onto the surface of the keratin materials, e.g., at least one zone of the skin.

In the application, unless specifically mentioned otherwise, contents, parts and percentages are expressed on a weight basis.

Other than in the operating examples, or where otherwise indicated, all numbers expressing quantities of components and/or reaction conditions are to be understood as being modified in all instances by the term “about”, with conventionally known meaning in the art, e.g., within 10% of the indicated number (e.g. “about 10%” means 9%-11% and “about 2%” means 1.8%-2.2%).

In the application, a numeric range is defined with a lower and an upper limits when needed, such as one or more lower limits and one or more upper limits are given to form a range optionally along with one ore more preferred ranges. A given range can be defined by selecting a lower limit and an upper limit that define the boundaries of the given range. All ranges defined in this manner are inclusive and combinable, that is, any lower limit can be combined with any upper limit to form a range, as long as the lower and upper limits are provided to modify the same subject. For example, when ranges of 60-110 and 80-120 are listed for a specific subject, it is to be understood that ranges 60-120 and 80-110 are also contemplated and encompassed. Further, if the lower limits listed are 1 and 2 and the upper limits are listed as 3, 4 and 5, the following ranges are all predictable and within the scope of the present disclosure: 1-3, 1-4, 1-5, 2-3, 2-4 and 2-5.

For the purposes of the present invention, the term “anhydrous” means absence of water, or the presence of water in such an amount those skilled can determine it as free or substantially free of water. For example, an “anhydrous” composition according to the present invention may comprise less than 2%, preferably less than 0.5% by weight of water relative to the total weight of the composition. Where appropriate, such small amounts of water may be provided by ingredients of the composition that comprise it in residual amount, but are not deliberately provided.

The present invention is directed to a composition for caring for keratin materials, comprising, consisting essentially of, or consisting of the components of:
A) a component comprising polydatin; and
B) at least two polyols, wherein at least one of the polyols is a C2-C6 diol,
wherein the composition is anhydrous.

Component A) Comprising Polydatin

Polydatin usually refers to 3,4′,5-trihydroxystilbene-3-beta-monoglucoside, in glycoside form of resveratrol with a monocrystalline structure which is isolated from the bark of Picea sitchensis or Polygonum cuspidatum. Polydatin may be detected in grape, peanut, hop cones, red wines, hop pellets, cocoa-containing products, chocolate products and many daily diets.

Polydatin can be particularly obtained from the root of Polygonum cuspidatum. Thus, for the purpose of the present invention, a Polygonum cuspidatum root extract may be directly used as the component A). For example, a Polygonum cuspidatum root extract comprising 97 wt % or more polydatin can be directly used as the component A).

In some embodiments, the term Polygonum cuspidatum root extract can be used exchangeable with the term polydatin.

According to an embodiment, component A) consists essentially of, or consists of, polydatin.

The component A) may be present in an amount of 0.01-20% by weight, preferably 0.05-18% by weight, or 0.1-15% by weight, or 0.1-9% by weight, relative to the total weight of the composition.

Resveratrol

The composition according to the present invention may comprise resveratrol, in particular when a Polygonum cuspidatum root extract is directly used as the component A). That is, the resveratrol, if any, is generally present in component A).

According to an embodiment of the invention, for the component A), resveratrol and polydatin are used at a ratio by weight of 0.001:1 to 0.2:1, preferably 0.01:1 to 0.1:1.

Component B), Polyol

The composition according to the present invention comprises a polyol, which is liquid at ambient temperature (25° C.), preferably selected in particular from polyols having in particular from 2 to 20 carbon atoms, preferably having from 2 to 10 carbon atoms, and preferentially having from 2 to 6 carbon atoms, such as glycerin, propylene glycol, e.g. 1,3-propylene glycol or 1,2-propylene glycol, butylene glycol, pentylene glycol, hexylene glycol, dipropylene glycol, and diethylene glycol.

Preferably, the composition according to the present invention may comprise concurrently at least two polyols. According to an embodiment of the invention, for the at least two polyols concurrently used, at least one polyol is a diol, preferably a C2-C6 diol, e.g., a C2-C4 diol.

According to an embodiment of the invention, for the at least two polyols concurrently used, at least two of, preferably all of, the polyols are lower alcohols, preferably each having from 2 to 6 carbon atoms.

According to an embodiment of the invention, for the at least two polyols concurrently used, at least two polyols are each diols, preferably each having from 2 to 6 carbon atoms.

According to an exemplary embodiment of the invention, for the at least two polyols concurrently used, at least two of, preferably all of, the polyols are each derivatives of a same alkane. For example, a combination of ethylene glycol and diethylene glycol, which are deemed as derivatives of ethane, can be used. For another example, a combination of two, three, or four of propylene glycol, dipropylene glycol, glycerin, and diglycerin which are each deemed as derivatives of propane, can be used.

Examples of at least two polyols concurrently used can be made to a combination of propylene glycol and diprpyelene glycol; a combination of propylene glycol and glycerin; a combination of glycerin and diglycerin; a combination of propylene glycol, dipropylene glycol, and glycerin; and a combination of propylene glycol, dipropylene glycol, glycerin, and diglycerin.

According to an embodiment of the invention, the component A) and the component B) are used in a ratio by weight of 0.0001:1 to 0.3:1, preferably 0.0005:1 to 0.25:1, or preferably 0.001:1 to 0.22:1.

According to an embodiment of the invention, for the polyol used for component B), at least one diol, preferably a C2-C6 diol, e.g., propylene glycol, can represents a significant content thereof, preferably the highest amount compared with any other polyols. For example, for the component B), at least one diol, preferably a C2-C6 diol, e.g., propylene glycol or a combination of two or more diols, can represents 40-80% by weight, preferably more than 50 wt % up to 80 wt %, or up to 70 wt %, e.g., 60-70% by weight, relative to the total weight of the component B). For another example, for the component B), a C2-C4 diol represents the highest amount compared with any other polyols, e.g., 40-60% by weight, relative to the total weight of the component B).

Anhydrous Form

For the purpose of the present invention, the composition according to the present invention is essentially in an anhydrous form. According to the present invention, water or moisture is preferably avoided to be included in the composition; however, it is easily understood by those skilled that water may not be 100% removed. For example, although water is not intentionally added, it may be provided by ingredients of the composition that contain it in residual amount, may be incorporated during the production, or may be absorbed from ambient during production and/or storage.

Accordingly, for the purposes of the present invention, the term “anhydrous” means absence of water, or the presence of water in such an amount that those skilled can determine it as free of or substantially free of water. For example, an “anhydrous” composition according to the present invention may comprise less than 2%, preferably less than 0.5% by weight of water, relative to the total weight of the composition. Preferably, an “anhydrous” composition according to the present invention does not comprise a detectable amount of water, wherein the “detectable amount” means an amount that can be detected by a device conventionally used in the art to measure the water content.

Optionally, the components A) and B), as well as, if present, any other components, are subjected to a water removal step before being incorporated into the composition of the present invention. The technical means for the water removal is not particularly limited, as long as the composition obtained is “anhydrous”.

Antioxidants

The composition according to the present invention can optionally comprise an antioxidant.

The antioxidants used may include natural exogenous phytochemical antioxidants such as phenols and carotenoids.

The antioxidant can include flavonoids. Flavonoids constitute a large class of more than 5,000 polyphenolic phytochemicals with antioxidant properties that act by direct free radical scavenging. Flavonoids have anti-inflammatory, antibacterial, antiviral, anti-allergic, anti-mutagenic, anti-thrombotic, anti-tumor and vasodilating effects and these methods of action can also be used to prevent, alleviate or eliminate oxidative damage from dental instruments. Flavonoids also exhibit chelation properties with metal ions and can mitigate oxidative damage from metal ions by chelating ions. The formation and stability of flavonoid-metal chelate is dependent on the function of the structure. Flavonoids having a catechol moiety and having a hydrogen bond between the hydroxyl groups at the 5-position and the 3-position have chelation properties.

Vitamin C and derivatives can be used, including ascorbic acid, sodium ascorbate and the fat-soluble ester tetrahexyl decyl ascorbate and ascorbyl palmitate, magnesium ascorbyl phosphate, ascorbyl glucoside, glucosamine ascorbate, ascorbyl acetate, and the like. In addition, it is also possible to use plants derived from a large amount of vitamin C, such as extracts of Myrciaria dubia, acerola, Emblica officinalis, and bioflavonoids from rose hips and citrus, including Water-soluble bioflavonoids e.g., hesperidin methyl chalcone.

Sesamum indicum or lignan may also be added. Sesame and its lignans (fibrous compounds associated with sesame) act as antioxidants. Sesame seed lignan significantly enhances vitamin E activity.

Other antioxidants which may be incorporated into the compositions of the present invention include tocopherols (such as d-alpha-tocopherol, d-beta-tocopherol, d-gamma-tocopherol, d-delta-tocopherol), tocotrienol Phenol (eg d-α-tocotrienol, d-β-tocotrienol, d-γ-tocotrienol, d-δ-tocotrienol) and vitamin E (α-tocopheryl acetate)). These compounds can be isolated from natural sources, prepared by synthetic means or mixed. The tocotrienol-rich vitamin E preparation can be obtained by fractionating the vitamin E preparation to remove a portion of the biophenol and recovering the higher concentrated tocotrienol product. Useful tocotrienols are natural products isolated, for example, from wheat germ oil, grain or palm oil using high performance liquid chromatography or from barley, distiller's grains or oats by alcohol extraction and/or molecular distillation. The term “tocotrienol” as used herein includes a tocotrienol-rich fraction obtained from these natural products as well as a pure compound. Increased glutathione peroxidase activity protects the skin from oxidative damage.

In addition, carotenoids, especially lutein types, are also useful antioxidants that can be used. Lutein-type carotenoids include molecules such as lutein, canthaxantin, cryptoxanthin, zeaxanthin and astaxanthin. Lutein compounds protect compounds such as vitamin A, vitamin E and other carotenoids.

The flavonoid may be a flavanone (a derivative of 2,3-dihydro-2-phenylbenzopyran-4-one). Flavanones include: scutellarin, eriodictin, hesperetin, hesperidin, sylvestre, isosakuranetin, naringenin, naringin, pinocin, tangrin (poncirin)), sakuranetin, sakura glycosides and 7-O-methyl ergophenol (Sterubin).

The flavonoid may be a dihydroflavonol (a derivative of 3-hydroxy-2,3-dihydro-2-phenylbenzopyran-4-one). Flavanols include: taxifolin, Aromadedrin, Chrysandroside A, Chrysandroside B, Xeractinol, astilbin, and flavonol.

The flavonoid may be a flavonoid (a derivative of 2-phenylbenzopyran-4-one). Flavonoids include: Apigenin, luteolin, tangeritin, Chrysin, baicalein, wild baicalein, wogonin, synthetic flavonoids: Diosmin and flavonoids ester.

The flavonoid may be a flavonol (a derivative of 3-hydroxy-2-phenylbenzopyran-4-one). Flavonols include: 3-hydroxyflavone, rhodoxanthin, quercetin, galangin, cotton dermatan, kaempferol, kaempferol, isorhamnetin, mulberry pigment, myricetin, naringin (Natsudaidain), Muskyl flavonol (Pachypodol), quercetin, methyl rhamnosin, rhamnetin, azalein, hyperoside, isoquercetin, kaempferol, myricetin, suede Glycosides, Robinin, Rutin, Spiraea, Xanthorhamnin, Amurensin, Icariin and Tracuridine.

The flavonoid may be a flavan-3-ol (a derivative of 2-phenyl-3,4-dihydro-2H-benzopyran-3-ol). Flavan-3-ol includes: catechin, epicatechin, epigallocatechin, epicatechin gallate, epigallocatechin gallate, epiafzelechin, Fisetinidol, Guibourtinidol, Mesquitol and Robinetinidol.

The flavonoid may be a flavan-4-ol (a derivative of 2-phenylchroman-4-ol). Flavan-4-ols include: Apiforol and Luteoforol.

The flavonoid may be an isoflavone (a derivative of 3-phenylbenzopyran-4-one). Isoflavones include: genistein, daidzein, garbanin A, formononetin, and equol metabolites from daidzein.

The antioxidant may be anthocyanin (a derivative of 2-phenylbenzopyranoside cation). Anthocyanins include: Aurantinidin, cyanidin, delphinidin, Europinidin, Luteolinidin, Pelargonidin, Malvidin, Peonyidin (Peonidin)), morning glory pigment (Petunidin), rose pigment (Rosinidin) and xanthone.

The antioxidant may be dihydrochalcone (a derivative of 1,3-diphenyl-1-propanone). Dihydrochalcone includes: phloretin, dihydrochalcone phloridin cisplatin, Aspalathin, naringin dihydrochalcone, neohesperidin dihydrochalcone and Nothofagin. The mode of action of the present invention is not limited, but dihydrochalcone can exert an antioxidant effect by reducing active radicals such as active oxygen and reactive nitrogen species.

The antioxidant can be anthocyanin. Anthocyanins and their derivatives are antioxidants. Anthocyanins comprise a class of flavonoid compounds responsible for the red, purple and blue colors of many fruits, vegetables, grains and flowers, which are naturally occurring water-soluble compounds. In addition, anthocyanins are collagenase inhibitors. Inhibition of collagenase helps prevent and reduce wrinkles caused by skin collagen reduction, increase skin elasticity, and the like. Anthocyanins can be obtained from any part of a variety of plant sources, such as solids, flowers, stems, leaves, roots, bark or seeds. Those skilled in the art will appreciate that certain portions of the plant may comprise higher natural levels of anthocyanins, and thus these moieties are used to obtain the desired anthocyanins. In some cases, the antioxidant can include one or more betaine. Betatin, similar to anthocyanins, is available from natural sources and is an antioxidant.

The antioxidant may be a phenylpropanoid (a derivative of cinnamic acid). Phenylpropanoids include: cinnamic acid, caffeic acid, ferulic acid, trans-ferulic acid (including its antioxidant pharmacore 2,6-dihydroxy acetophenome), 5-hydroxyferic acid, sinapic acid, Coumarin, coniferyl alcohol, sinapyl alcohol, eugenol, Chavicol, baicalein, P-coumaric acid and sinapinic acid. Without limiting the mode of action of the present invention, phenylpropanoids can neutralize free radicals.

The antioxidant may be chalcone (a derivative of 1,3-diphenyl-2-propen-1-one). Chalcone includes: zirconia, Okanin, safflower, Marein, Sophoradin, Xanthohumol, Flavokvain A, Flavokavain B, Flavokavin C and Synthetic Safalcone.

The antioxidant may be curcuminoid. Curcuminoids include: curcumin, demethoxycurcumin, bis-demethoxycurcumin, tetrahydrocurcumin, and tetrahydrocurcumin. Curcumin and tetrahydrocurcumin can be derived from the rhizome of turmeric. Tetrahydrocurcumin, a metabolite of curcumin, has been found to be a more potent antioxidant and more stable than curcumin.

The antioxidant can be tannin. Tannins include: tannins, Terflavin B, Glucogallin, Dgallic acid, and Quercitannic acid.

The antioxidant can be a stilbenoid. The mites include: resveratrol, red sandalwood and paclitaxel. Resveratrol can include, but is not limited to, 3,5,4′-trihydroxyindole, 3,4,3′,5′-tetrahydroxyindole (cetotriol), 2,3′,4,5′-Tetrahydroxyindole (oxidized resveratrol), 4,4′-dihydroxyindole and its alpha and beta glucoside, galactoside and mannoside derivatives.

The antioxidant may be coumarin (a derivative of 2H-benzopyran-2-one). Coumarins include: 4-hydroxycoumarin, umbelliferone, Aesculetin, Herniarin, Auraptene, and dicoumarin.

The antioxidant can be a carotenoid. Carotenoids include: beta-carotene, alpha-carotene, gamma-carotene, beta-cryptoxanthin, lycopene, lutein and idebenone.

The antioxidant can be a vitamin. Vitamins include: retinol, ascorbic acid, L-ascorbic acid, tocopherol, tocotrienol and vitamin cofactor: coenzyme Q10.

The anti oxidant may be: xanthone, butylated hydroxytoluene, 2,6-di-tert-butylphenol, 2,4-dimethyl-6-tert-butylphenol, gallic acid, eugenol, uric acid, α-lipoic acid, ellagic acid, cichoric acid, chlorogenic acid, rosmarinic acid, salicylic acid, acetylcysteine, S-allylcysteine, pyridone (Barbigerone), [symbol] Chebulagic acid, edaravone, ethoxyquin, glutathione, hydroxytyrosol, idebenone, melatonin, N-acetyl serotonin, nordihydroguaiac Acid, Oleotanthal, oleuropein, Paradol, paclitaxel, probucol, propyl gallate, protocatechuic acid, pyrithione, rutin, flax lignan diglucoside, sesamin, sesame phenol, Silybin, silymarin, theaflavins, theaflavins digallate, Thmoquinone, Trolox, tyrosol, polyunsaturated fatty acids and sulfur-based antioxidants such as methionine or lipoic acid.

Vitamin C and derivatives can be used, including ascorbic acid, sodium ascorbate and the fat-soluble ester tetrahexyl decyl ascorbate and ascorbyl palmitate, magnesium ascorbyl phosphate, ascorbyl glucoside, glucosamine ascorbate, ascorbyl acetate, and the like. In addition, it is also possible to use plants derived from a large amount of vitamin C, such as extracts of Myrciaria dubia, acerola, Emblica officinalis, and bioflavonoids from rose hips and citrus, including Water-soluble bioflavonoids such as hesperidin methyl chalcone.

Sesamum indicum or sesame lignan can also be used. Sesame and its lignans (fibrous compounds associated with sesame) act as antioxidants. Sesame seed lignan significantly enhances vitamin E activity.

Other antioxidants which may be incorporated into the compositions of the present invention include tocopherols (such as d-alpha-tocopherol, d-beta-tocopherol, d-gamma-tocopherol, d-delta-tocopherol), tocotrienol Phenol (eg d-α-tocotrienol, d-β-tocotrienol, d-γ-tocotrienol, d-δ-tocotrienol) and vitamin E (α-tocopheryl acetate)). These compounds can be isolated from natural sources, prepared by synthetic means or mixed. The tocotrienol-rich vitamin E preparation can be obtained by fractionating the vitamin E preparation to remove a portion of the biophenol and recovering the higher concentrated tocotrienol product. Useful tocotrienols are natural products isolated, for example, from wheat germ oil, grain or palm oil using high performance liquid chromatography or from barley, distiller's grains or oats by alcohol extraction and/or molecular distillation. The term “tocotrienol” as used herein includes a tocotrienol-rich fraction obtained from these natural products as well as a pure compound. Increased glutathione peroxidase activity protects the skin from oxidative damage.

In addition, carotenoids, especially lutein types, are also useful antioxidants that can be used. Lutein-type carotenoids include molecules such as lutein, canthaxantin, cryptoxanthin, zeaxanthin and astaxanthin. Lutein compounds protect compounds such as vitamin A, vitamin E and other carotenoids.

The antioxidant described above can be present according to the invention in an amount ranging from 0.001 to 15% by weight, preferably from 0.1 to 7% by weight, or preferably from 0.5 to 5% by weight, relative to the total weight of the composition of the present invention.

Preservative

For use of the present invention, the composition according to the present invention may optionally comprise a preservative. The preservative useful according to the present invention can be any one conventionally used for cosmetics, in particular for ampoule products.

For example, preservatives can be used according to the present invention comprise methylchloroisothiazolinone, imidazolidinyl urea, derivatives of hydantoin, such as DMDMH, parahydroxybenzoate ester, phenoxyethanol, benzyl alcohol, chlorphenesin, benzoic acid and a salt thereof, such as sodium benzoate, potassium sorbate, hydroxyacetophenone, amino-acid based preservative, sorbitan octanate, glycerol caprylate and the like.

The preservative can be present according to the invention in an amount 0.01-5%, preferably 0.01%-3%, preferably 0.01%-1%, relative to the total weight of the composition of the present invention.

Gelling Agent

As gelling agents, mention may be made of carboxyvinyl polymers such as Carbopols® (Carbomers) and Pemulen such as Pemulen TR1® and Pemulen TR2® (acrylate/C10-C30-alkylacrylate copolymer); polyacrylamides, for example crosslinked copolymers sold under the names Sepigel 305® (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80) by the company Seppic; polymers and copolymers of 2-acrylamido-2-methylpropanesulphonic acid, optionally crosslinked and/or neutralized, such as the polyacid 2-acrylamido-2-methylpropanesulphonic sold by Hoechst under the trade name “Hostacerin AMPS®” (CTFA name: ammonium polyacryloyldimethyl taurate or SIMULGEL 800® marketed by the company SEPPIC (CTFA name: sodium polyacryolyldimethyl taurate/polysorbate 80/sorbitan oleate); copolymers of 2-acrylamido-2-methylpropanesulphonic acid and hydroxyethyl acrylate such as SIMULGEL NS® and SEPINOV EMT 10® marketed by the company SEPPIC, cellulose derivatives such as hydroxyethylcellulose, polysaccharides and especially gums such as Xanthan gum, guar gum or carob, water-soluble or water-dispersible silicone derivatives such as acrylic silicones, polyether silicones and cationic silicones and rs mixtures.

In a particularly preferred embodiment, the composition according to the invention comprises at least one optionally crosslinked and/or neutralized 2-acrylamido-2-methylpropanesulfonic acid polymer and copolymer, such as polyacrylic acid 2-acrylamido-2-methylpropanesulfonic acid and or at least one carboxyvinyl polymer such as carbomers. Preferably, the composition according to the invention comprises the polyacid 2-acrylamido-2-methylpropanesulphonic sold by the company Hoechst under the trade name “Hostacerin AMPS®” (CTFA name: ammonium polyacryloyldimethyltaurate) and at least one carboxyvinyl polymer such as the Carbopols® (Carbomers).

The gelling agents may be present in the composition according to the invention in particular in an amount of 0.1% to 3% by weight, or particularly from 0.5% to 2% by weight, relative to the total weight of the composition.

Adjuvant

In a known manner, the dosage forms dedicated to topical application may also comprise adjuvants which are customary in the cosmetic, pharmaceutical and/or dermatological field, such as gelling agents, hydrophilic or lipophilic active agents, fragrances, fillers, filters, pH adjusters, odor absorbers and dyes. The amounts of these various adjuvants are those conventionally used in the field under consideration, and for example from 0.01 to 20% of the total weight of the composition. These adjuvants, depending on their nature, can be introduced into the aqueous phase.

These adjuvants and their concentrations must be such that they do not modify the desired property for the composition of the invention.

Additional Active Ingredient

The composition according to the invention may advantageously comprise one or more additional active ingredients, in particular cosmetic, dermatological or pharmaceutical.

In the context of the present invention, an additional active ingredient is an active ingredient other than polydatin or resveratrol present in the composition and the other active agents specifically defined above.

Advantageously, such additional active ingredient, cosmetic, dermatological or pharmaceutical, may be intended to exert a cosmetic, care or hygiene effect on keratin materials e.g., skin, including face, hair, eyelashes, scalp and/or leather hairy, and preferentially on the skin.

The choice of this additional active ingredient is of course conditioned by the effect sought jointly by the composition in question.

Thus, a composition according to the invention may further comprise an active ingredient having an anti-aging activity, or a hydration activity.

Of course, those skilled in the art will take care to choose the optional additional active ingredient (s) added to the composition according to the invention in such a way that the advantageous properties intrinsically attached to the composition according to the invention are not, or substantially not, altered by the proposed addition.

These additional active ingredients are generally present in the composition in a content ranging from 0.0001% to 20% by weight and preferably from 0.01% to 10% by weight relative to the total weight of said composition.

The compositions according to the invention can be manufactured by known processes, generally used in the cosmetic or dermatological field.

The composition according to the invention finds its application in a large number of treatments for keratin materials such as the skin, the scalp or the mucous membranes (lips), and more particularly the skin, in particular in the care of the skin, to nourish the skin, prevent the loss of transepidermal water and/or protect the skin.

The subject of the invention is also a process for the care or makeup of keratin materials, comprising the application to the keratin materials of a composition as defined above.

In particular, the subject of the invention is a process for treating dry and/or rough skin, comprising the application to the skin of a composition as defined above.

In particular, the subject of the invention is a process for treating aged skin, comprising the application to the skin of a composition as defined above.

The invention also relates to the use of a composition as defined above, for the care or makeup of keratin materials.

In a particular embodiment, the composition is suitable for the treatment of dry skin.

In a particular embodiment, the composition is suitable for the treatment of aged skin.

In a particular embodiment, the composition according to the present invention shows particularly an improved anti-singlet oxygen effect.

### Examples

The following examples of compositions according to the invention are given by way of illustration and without limitation. The compounds are indicated in chemical name or INCI name.

The ingredient amounts/concentrations in the compositions/formulas described below are expressed in % by weight, relative to the total weight of each composition/formula, unless otherwise indicated.

TABLE 1
Compositions of Example 1 of the Invention
and Comparative Examples 1-2;

| | | Content | |
|---|---|---|---|
| | | (% by weight) | |
| Chemical or INCI name | Ex. 1 | C. Ex. 1 | C. Ex. 2 |
| GLYCERIN | 32.3% | 20.17% | 30.9% |
| DIPROPYLENE GLYCOL | 19% | 19% | 19% |
| PROPANEDIOL | 48.2% | 48.2% | 48.2% |
| POLYGONUM CUSPIDATUM ROOT | 0.5% | | |
| EXTRACT (available from GUILIN | | | |
| LAYN NATURAL INGREDIENTS) | | | |
| ASCORBIC ACID | | 12.63% | |
| FERULIC ACID | | | 1.9% |

The procedures of preparing the compositions in above examples were briefly introduced as below:
1). weighing all the ingredients and mixing them together;
2). stirring the ingredients at a temperature of 80° C. until all dissolved; and
3). cooling to room temperature.

Evaluation

Evaluation of the anti-oxidation ability of the compositions.

For each composition the anti-oxidation ability was evaluated by a SQUALENE TEST (thin layer), comprising:
a. preparing 1% squalene ethanol solution;
b. preparing squalene film on ground glass slides, with around 50 μg/cm²;
c. applying the composition on squalene film, with 9 repeat slides for one composition;
d. stressing “solar” irritation on each prepared slides (step c) for 5 j/cm² of UVA in total (calculated by irritation strength at 360 nm);
e. extracting the oxidized squalene peroxides and squalene residue with methanol on a lab shaker; and
f. quantify the squalene peroxides and squalene extracted.

Results were provided below:

TABLE 2
Performance of the compositions

| Properties | Ex. 1 | CE. 1 | CE. 2 |
|---|---|---|---|
| per-oxidized squalene % | 41% | 64% | 59% |
| IN-VITRO ANTIOXIDATION | | | |
| SQUALENE TEST (THIN LAYER) | | | |

Note:
the less per-oxidized sebum %, the better the anti-oxidation ability was.

1: A composition for caring for keratin materials, comprising:
A) a component comprising polydatin; and
B) at least two polyols, wherein at least one of the polyols is a C2-C6 diol,
wherein the composition is anhydrous.

2: The composition according to claim 1, wherein the polydatin comprises a Polygonum cuspidatum root extract.

3: The composition according to 11, wherein the component A) is present in an amount of 0.01-20% by weight, preferably 0.05-18% by weight, or preferably 0.1-15% by weight, relative to the total weight of the composition.

4: The composition according to claim 1, wherein the component A) further comprises resveratrol.

5: The composition according to claim 1, wherein the component B) is selected from polyols having from 2 to 20 carbon atoms.

6: The composition according to claim 1, wherein at least two of the polyols are lower alcohols having from 2 to 6 carbon atoms.

7: The composition according to claim 1, wherein at least two polyols are each diols, having from 2 to 6 carbon atoms.

8: The composition according to claim 1, wherein for the component B), a C2-C4 diol represents 40-60% by weight, relative to the total weight of the component B).

9: The composition according to claim 1, wherein the component A) and the component B) are used in a ratio by weight of 0.0001:1 to 0.3:1.

10: The composition according to claim 1, wherein the composition comprises less than 2% by weight of water, relative to the total weight of the composition.

11: The composition according to claim 1, wherein the composition comprises an antioxidant selected from a natural exogenous phytochemical antioxidant, flavonoid, vitamin, tocopherols, tocotrienol phenol, carotenoid, anthocyanin, dihydrochalcone, phenylpropanoid, chalcone, curcuminoid, tannin, stilbenoid, coumarin, carotenoid, or a mixture thereof.

12: The composition according to claim 1, wherein the antioxidant is used in an amount of from 0.001 to 10% by weight relative to the total weight of the composition.

13: The composition according to claim 1, wherein the composition further comprises a preservative.

14: A method for caring for keratin materials, comprising application of the composition according to claim 1 to the keratin material.

15: The method according to claim 14, wherein the keratin material is skin and the application is topical.

16: The composition according to claim 1 wherein the component B) is selected from the group consisting of glycerin, propylene glycol, butylene glycol, pentylene glycol, hexylene glycol, dipropylene glycol, and diethylene glycol.

17: The composition according to claim 11, wherein the antioxidant is a vitamin selected from the group consisting of vitamin C, vitamin E and derivatives thereof.

18: The composition according to claim 1 wherein the composition comprises less than 0.5% by weight of water, relative to the total weight of the composition.
